(19) United States
(12) Patent Application Publication
(10) Pub. No.: US 2024/0399030 A1
(43) Pub. Date: Dec. 5, 2024

(54) Modified Arthroscopic Tissue Engineered Total Hip

(71) Applicant: James M. Weiss, Germantown, MD (US)

(72) Inventor: James M. WEISS, Germantown, MD (US)

(21) Appl. No.: 18/674,539

(22) Filed: May 24, 2024

Related U.S. Application Data
(60) Provisional application No. 63/504,111, filed on May 24, 2023.

(51) Int. Cl. A61L 27/58; A61B 17/00; A61B 17/16; A61F 2/46; A61L 27/04; A61L 27/12; A61L 27/22; A61L 27/36; A61L 27/54

(52) U.S. Cl. CPC A61L 27/58; A61B 17/1668; A61F 2/4601; A61F 2/4607; A61F 2/4609; A61F 2/4675; A61L 27/047; A61L 27/12; A61L 27/225; A61L 27/3604; A61L 27/54; A61B 2017/00557; A61L 2300/216; A61L 2300/252; A61L 2300/414; A61L 2400/06

(57) ABSTRACT

Disclosed is an implantable joint replacement device. The device includes an elongated body having a medial component and a lateral component. The device includes an internal channel formed inside the elongated body, the internal channel extending along a portion of the elongated body. The device includes an inlet adjacent to the lateral component of the elongated body, the inlet being in fluid communication with the internal channel. The device includes an outlet disposed along a surface of the elongated body, the outlet being in fluid communication with the internal channel.

### CROSS-REFERENCE TO RELATED APPLICATIONS

This application claims the benefit of U.S. Provisional Application No. 63/504,111, filed May 24, 2023, which is hereby incorporated in its entirety by reference.

### BACKGROUND

Hip arthroplasty has been described going back to the early part of the 20th century. The advent of the modern era of hip replacement surgery was initially described by Sir John Chamley who published his initial series of cases in the Lancet in 1962. Over the past five decades improvements in material science and a better understanding of the modes of failure of hip replacement has led to more predictable surgical outcomes and greater durability of implants. Despite these improvements joint implants still have significant short and long-term morbidities associated with their implantation and impose activity restrictions on those who receive them.

The initial iteration of hip replacement implants originally described by Chamley and subsequently by others relied on polymethyl-methacrylate to secure them to the underlying bone. Good early results were obtained and led to wider adoption of the procedure. Midterm follow up showed that many of the implants became loose and failed at the cement bone or cement implant interface. Implants were developed in the 1980's and 1990's which incorporated interference or press fit designs and porous coatings to allow the implant to be affixed to the bone without the use of bone cement. However, premature failure and loosening of the devices were still seen and it was realized, as described by Harris et al. that submicroscopic particulate debris generated by polyethylene, the material used in cup liner, was the underlying source of the problem. It is postulated that this debris, in addition to third body wear, causes an inflammatory response in the effective joint space leading to the release of degradative enzymes which result in bone loss and implant loosening.

Consequently, significant research and effort has been directed at improving the wear couple between the femoral head and the acetabular component to minimize the production of wear debris as well as reducing the amount of third body wear. Ultra-high weight, highly cross-linked polyethylene has been developed to address this problem, and has shown a much lower degree of wear compared to its predecessor. However, subsurface stripping, brittleness and crack propagation and toughness have been an issue with this material. Ceramics, with their excellent wettability and hardness have shown good long term results in European studies with minimal wear debris and long term survival. However, edge loading and subsequent liner fracture remains a problem in acetabular components placed outside a very narrow range.

Metal on metal components, either in resurfacing procedures or in total hip replacements held out great promise in striking a balance between eliminating the use of polyethylene and avoiding the brittleness of ceramic components. Unfortunately, metal ion generation leading to elevated levels of chromium and cobalt in individuals receiving these implants has led to restrictions in their use in women of childbearing age. Further, metal-on-metal implants have also resulted in local inflammatory reactions (adverse local tissue reactions-ALTR or ALVAL) in a small set of patients leading to severe local tissue destruction and implant failure with un-reconstructable joints. This has led to product recalls, FDA intervention and multi-billion dollar lawsuits and settlements. Thus, the ideal orthopedic implant for the indication addressed in this patent as well all orthopedic indications would be able to be implanted with minimal soft tissue disruption, cause local tissue regeneration to address the anatomic defect, not result in inflammation, enhance the local healing environment through the release of cell mediators, and after the tissue was restored, would be broken down into byproducts readily absorbed and processed by the body through recognized pathways.

### SUMMARY

Disclosed are implantable joint replacement devices. In one embodiment, the device includes an elongated body having a medial component and a lateral component. The device includes an internal channel formed inside the elongated body, the internal channel extending along a portion of the elongated body. The device includes an inlet adjacent to the lateral component of the elongated body, the inlet being in fluid communication with the internal channel. The device includes an outlet disposed along a surface of the elongated body, the outlet being in fluid communication with the internal channel.

In some embodiments, an implantable joint replacement device includes an elongated body having a medial component and a lateral component. The device also includes an outer bioabsorbable balloon and an inner bioabsorbable balloon, both inner and outer balloons adjacent to the medial component of the elongated body, the outer balloon being provided over the inner balloon. The device further includes a first channel and a second channel, the first and second channels formed inside the elongated body and extending along a portion of the elongated body. The device further includes a first inlet and a second inlet, both adjacent to the lateral component of the elongated body, the first inlet being in fluid communication with the first channel and the second inlet being in fluid communication with the second channel. The device further includes a first outlet in fluid communication with the first channel, the first outlet being disposed adjacent to an external surface of the inner balloon and is configured to provide a first fluid between an internal surface of the outer balloon and the external surface of the inner balloon. The device further includes a second outlet in fluid communication with the second channel, the second outlet being disposed inside the inner balloon and configured to provide a second fluid inside the inner balloon.

### BRIEF DESCRIPTION OF THE DRAWINGS

FIG. 1 illustrates a coronal section taken through an arthritic hip joint after a guide wire has been inserted centrally through a femoral neck into a femoral head, in accordance with some embodiments.

FIG. 2 illustrates a drill that is passed up the femoral neck and through the femoral head creating a pilot hole, in accordance with some embodiments.

FIG. 3 illustrates a view of an intramedullary saw osteotomizing the femoral neck in situ, in accordance with some embodiments.

FIGS. 4A and 4B illustrate the removal of osteophytes using arthroscopic burs, in accordance with some embodiments.

FIGS. 4C and 4D illustrate an alternate method of removing the osteotomized femoral head, in accordance with some embodiments.

FIG. 5 illustrates a view of the femoral head removed and the acetabular cartilage being removed using arthroscopic burs, in accordance with some embodiments.

FIGS. 6A-6C show the acetabular component being delivered through the pilot and positioned, in accordance with some embodiments.

FIG. 7 shows the initial molding balloon being inflated in the joint space, in accordance with some embodiments.

FIG. 8 shows fibrin glue delivered and set in the joint space, in accordance with some embodiments.

FIGS. 9A-9E show operations of forming devices in the joint space, in accordance with some embodiments.

FIGS. 10A-10D show a procedure for forming an implantable joint replacement device, in accordance with some embodiments.

FIGS. 11A-11E show an alternate procedure for forming an implantable joint replacement device, in accordance with some embodiments.

FIG. 12 illustrates a view of a completed procedure for forming an implantable joint replacement device, in accordance with some embodiments.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

The figures (FIGs.) and the following description relate to preferred embodiments by way of illustration only. One of skill in the art may recognize alternative embodiments of the structures and methods disclosed herein as viable alternatives that may be employed without departing from the principles of what is disclosed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed device (or method) for purposes of illustration only. One skilled in the art will readily recognize from the following description that alternative embodiments of the devices and methods illustrated herein may be employed without departing from the principles described herein.

### Orthopedic Implants

The present disclosure relates to the field of orthopedic implants. Specifically, the device may be in the area of total hip replacement and may involve the use of a tissue-engineered implant that is designed to be placed surgically through minimally invasive techniques. The implant may be placed through a small incision and would be guided fluoroscopically and arthroscopically.

The device may be designed to perform a total hip replacement using only biologic materials without the use of modular, previously machined, large bore, static implants. The procedure may contemplate the use of a scaffolding material that is a flowable, settable, absorbable hydroxy-appetite that can be injected into a balloon or preformed space and then allowed to set into a desired neo-femoral head. An acetabular component may be made/implanted at the same surgical procedure if surgically indicated. The acetabular component may also be made from a flowable hydroxy-appetite material delivered into a potential space or may be fabricated from a pre-formed material which is consistent with a desired anatomy. The acetabular component may be guided into its desired position and fixed in position either under arthroscopic guidance or through the use of surgical navigation instruments. In either instance, a euthermic or minimally exothermic reaction may be anticipated. The material(s) may be loaded with cytokines or other desired substances to influence the desired tissue to be produced in-situ once the scaffolding has been delivered. The scaffolding may influence the production of the bone component of the newly formed hip joint by the addition of dexamethasone to the substrate. The cartilage pathway may be directed by the addition of TGFβ3.

In some embodiments, the present disclosure relates to methods and apparatus for performing a total hip replacement. The described methods may also be used for sub-categories of total hip replacements, e.g., partial hip replacement or arthroscopic methods of addressing chondral or osteo-chondral defects or deficiencies of a hip joint. Congenital deformities or post-traumatic defects may be addressed as well such as, but not limited to, femoral retroversion, slipped capital femoral epiphysis, femoroacetabular impingement on the femoral side, e.g., cam lesions, coxa vara, Perthes disease, coxa magna, osteonecrosis, coxa valga, femoral retroversion, mal- and non-union, Pipkin fractures and idiopathic chondrolysis.

### First Generation Device

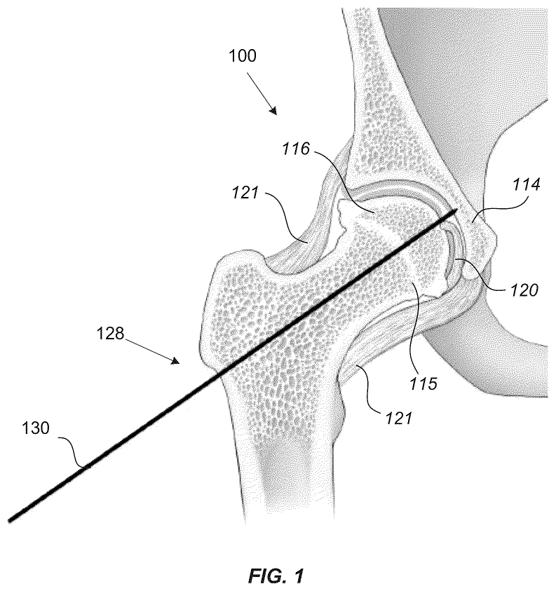

FIG. 1 illustrates a ball-and-socket hip joint 100. The socket is comprised of a cup-shaped cavity formed by the acetabulum 114, and the ball is formed by the head 116 of a femur. The head 116 projects from a neck 115 of the femur and is joined to the socket by a ligamentum teres attachment 120 and joint capsule 121.

Prior to performing a hip replacement procedure according to the present disclosure, an arthroscopy of the patient's ipsilateral knee may be performed about three weeks prior to the planned procedure. Using standard arthroscopic techniques, a harvest of a cartilage graft may be procured from the femoral condyle, where the non-articular portion of the femoral condyle is most accessible.

In one approach, to perform the hip replacement procedure, the patient is placed on a radiolucent fracture table in a supine position as would be typically used to address an intertrochanteric hip fracture. An incision is made at the inferior border of the greater trochanter in the midline of the lateral femur of the patient to expose a length of the lateral cortex 128 of the femur. Under fluoroscopic control, a guide wire 130 is passed centrally through the femoral neck 115 and head 116 (see FIG. 1). The guidewire is inserted under fluoroscopic guidance starting at the lateral cortex of the femur in the inter-trochanteric region, progressing up the center of the intact femoral neck, traversing the hip joint and docking in the acetabulum in the quadrilateral plate. Proper centering of the guide wire 130 may be confirmed on anteroposterior and/or lateral views. The anteroposterior and/or lateral views may be obtained by radiographic imaging.

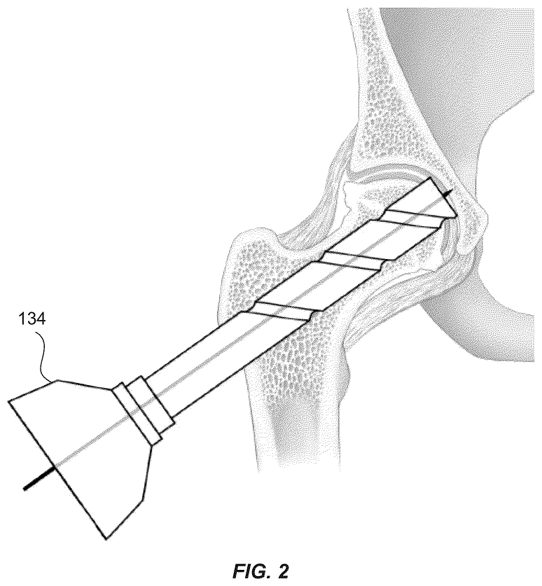

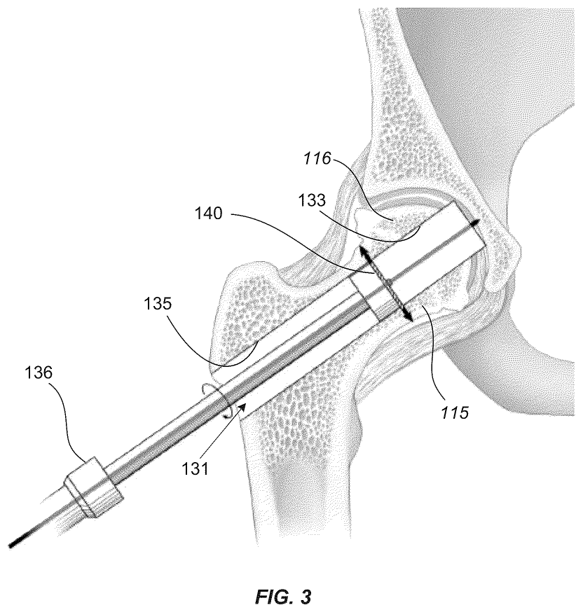

Once it has been confirmed that the guide wire 130 has been properly centered, a drill 134 is fed along the guide wire 130 to bore a pilot hole 131 through the femoral neck 115 and head 116 (see FIGS. 2 and 3). The drill may be passed under fluoroscopic control, drilling across the joint and stopping at the acetabulum. A front portion of the drill cuts a first diameter portion 133 of the pilot hole 131 (see FIG. 3), and a rear portion of the drill cuts a second diameter portion 135 of the pilot hole 131. In some embodiment, the first and second diameter portions may be the same. The first and second diameter portions may be equal to any diameter between 10-30 mm.

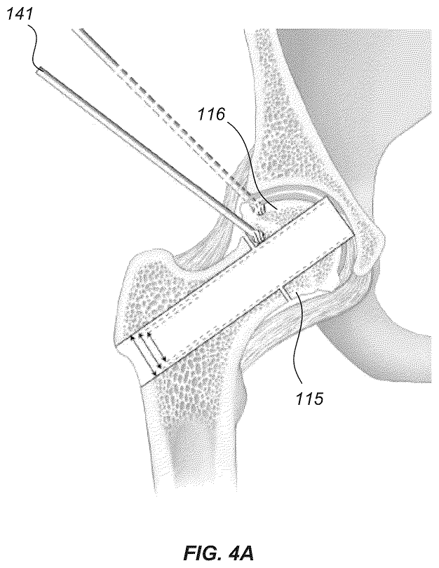

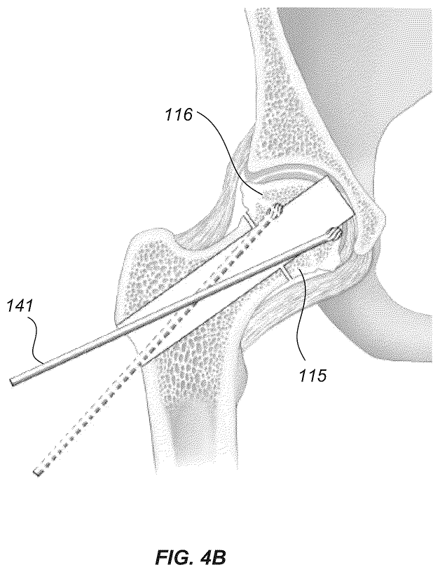

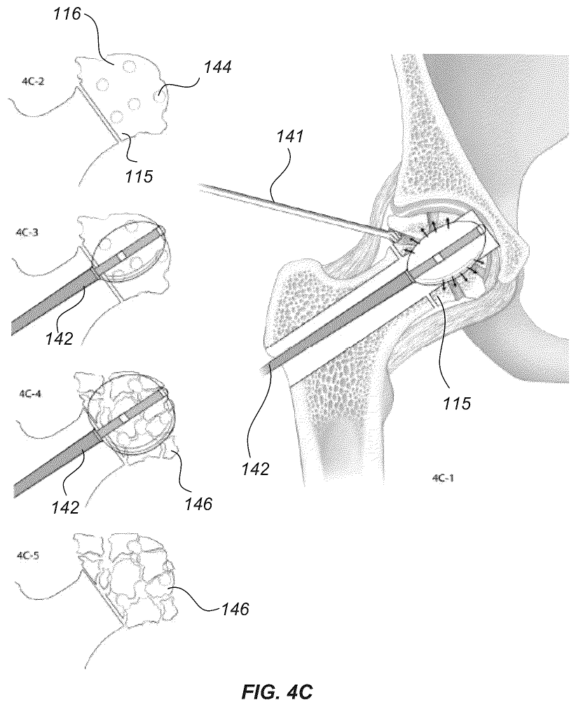

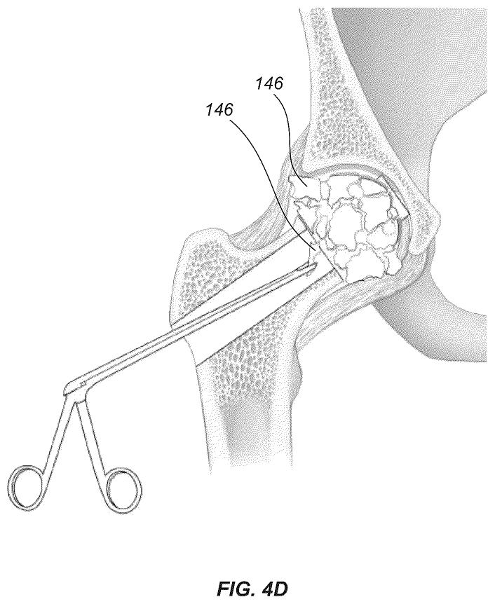

As shown in FIG. 3, a saw 136 is fed along the guide wire 130 until the blade 140 of the saw reaches the junction between the femoral neck 115 and head 116. This position may be confirmed by fluoroscopic views. The blade 140 of the saw 136 is then gradually deployed (e.g., under fluoroscopic guidance) until the neck 115 has been fully cut, disengaging (or osteotomizing) the femoral head 116 (see FIG. 4A). As a result, the head 116 is free floating in the joint except for its ligamentum teres attachment. Then, the guide wire 130 is removed. FIGS. 4A and 4B illustrate the removal of osteophytes using arthroscopic burs 141, in accordance with some embodiments.

FIGS. 4C and 4D illustrate an alternate method of removing the osteotomized femoral head by creating strategically placed stress risers in the femoral head using arthroscopic burs and exploding the femoral using a balloon. For example, the pieces 146 are show being removed through the pilot hole using a pituitary rongeur.

An arthroscope may be introduced into the hip joint or at the level of the osteotomy. A burr 141 may be used either arthroscopically and/or at the level of the resection or through a pilot hole to resect all or part of the femoral head depending on the desired level and amount of resection based on the underlying extent of the pathology (see 4C-1 of FIG. 4C). Alternatively, the femoral head could be removed by inserting an inflatable bone tamp 142 into the femoral head. Small stress risers 144 are created in the femoral head 116. The bone tamp is inserted into the femoral head 116 through a stress riser 144. The bone tamp 142 is then be inflated to fracture the femoral head 116 from inside out into small pieces 146 which can be removed (see FIGS. 4C and 4D).

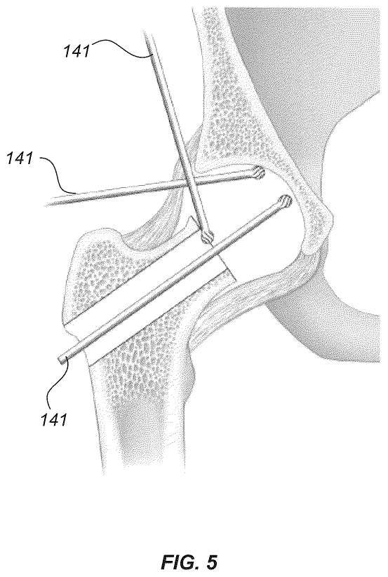

Once the bones of the femoral head are removed, a navigation system may be used to register points on the acetabular side. The remaining acetabular cartilage and any impinging osteophytes are removed. FIG. 5 illustrates a view of the femoral head and the acetabular cartilage being removed. For example, the acetabular component may be removed using arthroscopic burs 141 specific for this purpose (see FIG. 5) or a collapsible acetabular reamer may be designed for this purpose (not shown). The collapsible acetabular reamer may be passed through the pilot hole in its collapsed state and deployed and locked once it enters the potential joint space. The size of the acetabular component as well as the reamer may be predetermined base on pre-op templating studies. All labral pathology may also be addressed.

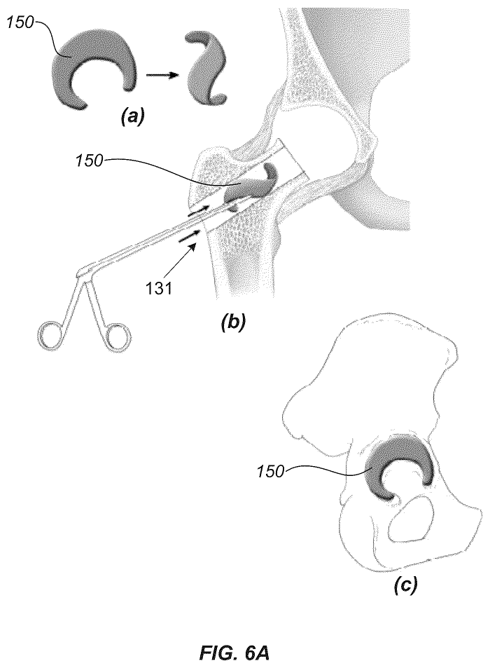

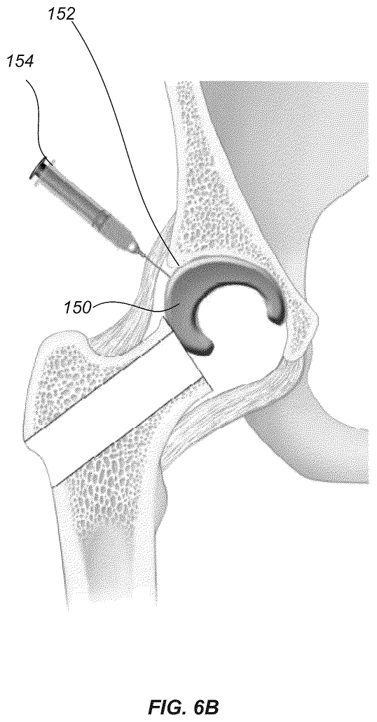

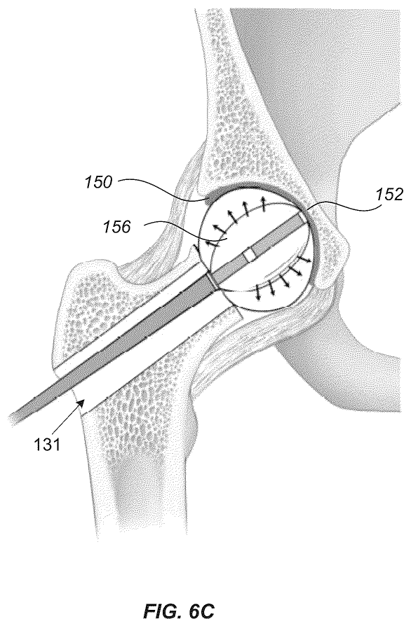

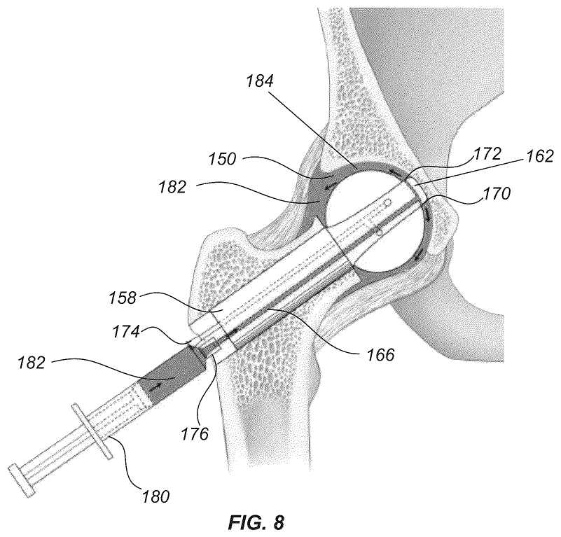

FIGS. 6A-6C show the acetabular component 150 being delivered through the pilot, positioned in the joint space, fibrin-glued into placed, and held by a balloon until it sets. The acetabular component 150, which has been pre-loaded with the appropriate cytokines, small molecules or other factors is then inserted through the pilot hole 131 and docked using the coordinates registered on the navigation system and with simultaneous arthroscopic confirmation (see FIGS. 6A (a), (b) and (c)). In some embodiments, it is anticipated that the acetabular component 150, by nature of its fabrication, will be semi-rigid in its characteristic. This may allow the acetabular component to be twisted on itself prior to insertion allowing it to pass through the pilot hole 131 and subsequently untwist itself or deploy once in the potential joint space (see FIG. 8). Once confirmed to be in the desired configuration and location the acetabular component 150 may be fixed in place with arthroscopic fixation devices and/or in combination with fibrin glue 152 or a similar adhesive. For example, FIG. 6B shows that a syringe 154 is used to apply fibrin glue 152 on the external surface of the acetabular component 150. In this iteration of acetabular reconstruction, an inflatable balloon 156 in the shape of the femoral head and the acetabulum is used to position the acetabular component 150 inside the joint space (see FIG. 6C). The inflatable balloon 156 is inserted through the pilot hole 131. The inflatable balloon 156 may be inflated with air, a saline solution, or any similar method. When inflated, the balloon 156 provides a support to help mold the acetabular component 150 to ensure a uniform surface texture and congruent surface visa-a-vis the subsequently newly formed femoral head. When maintained in position by the balloon 156, the fibrin glue 152 on the acetabular component 150 may dry within two or three minutes to position the acetabular component 150 on the internal surface of the space (or joint space) previously occupied by the femoral head. This process may be monitored arthroscopically.

In some embodiments, the acetabular component may also be formed by inserting a pre-formed mold through the pilot hole 131 and injecting a flowable, absorbable hydroxy-appetite material pre-loaded with cytokines or appropriate factors designed to allow local mesenchymal stem cells, which would be attracted to populate the material, to turn into cartilage. The mold may be removed after positioning the acetabular component.

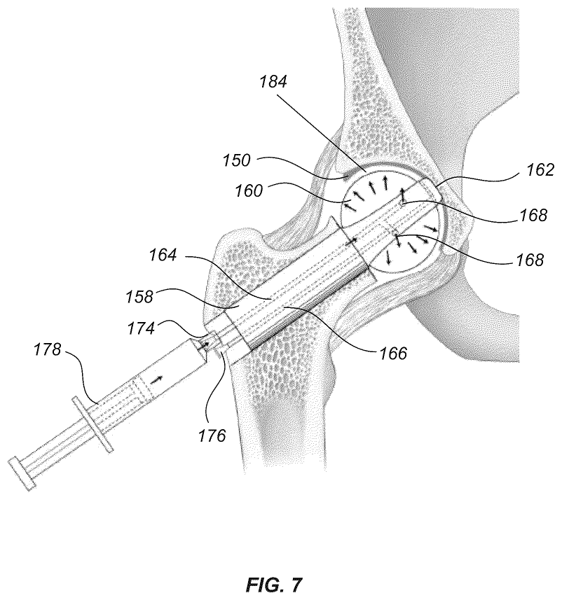

FIG. 7 shows a first forming device 158 with a smaller diameter balloon 160 compared the balloon 156. The purpose of the smaller diameter balloon 160 is to provide a space for fibrin glue to flow. The head 162 of the first forming device 158 may sit on the acetabulum fossa (or condyloid notch). The first forming device 158 has two channels 164 and 166 (or conduits). The first channel 164 communicates with a first inlet 174 and at least two first outlets 168. As shown in FIG. 7, a syringe 178 is connected to the first inlet 174 to provide a fluid (e.g., air, a saline solution, etc.) for inflating the balloon 160. The balloon 160 may be inflated to a diameter such that there is a gap 184 (e.g., 1 mm) between the balloon 160 and the acetabular component 150.

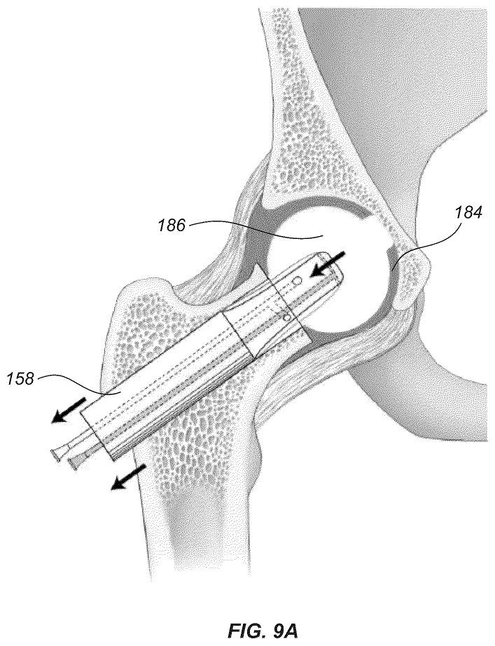

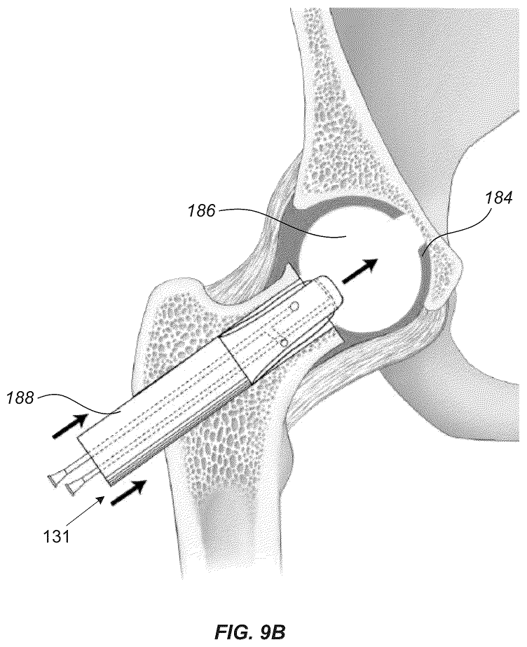

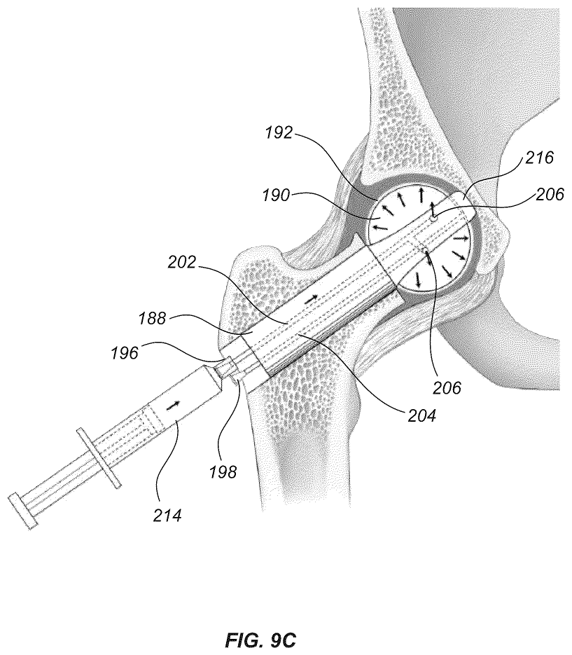

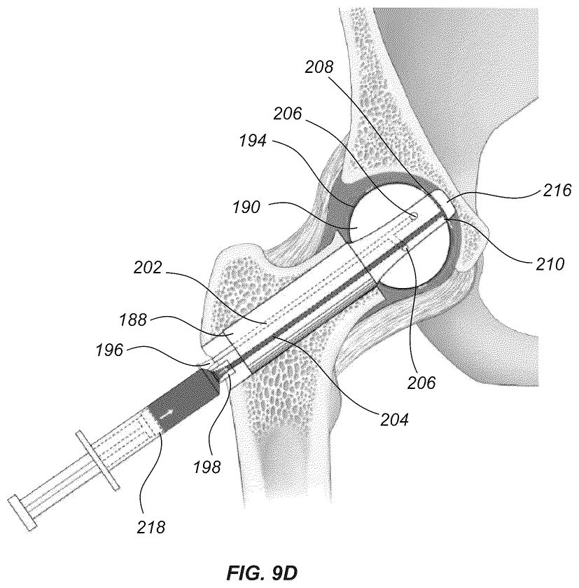

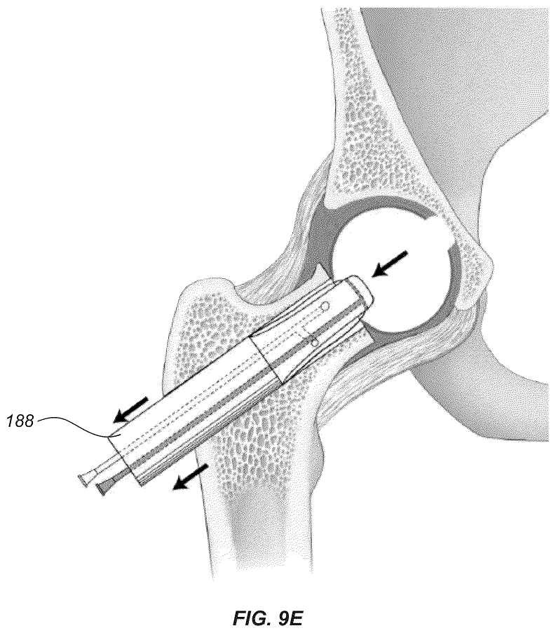

The fluid flows through the first channel 164 to exits at the outlets 168. The outlets 168 are located inside the balloons 160. The second channel 166 communicates with a second inlet 178 and second outlets 170 and 172. The outlets 170 and 172 are located adjacent to the external surface of the balloon 160 and adjacent to the head 162 as shown in FIG. 8. A syringe 180 may be connected to the second inlet 178 to provide fibrin glue 182 inside the joint space under fluoroscopy or arthroscopy. The fibrin glue 182 flows from the second inlet 178 into the second channel 166 to exit at the second outlets 170 and 172. The joint space may be filled with the fibrin glue 182. The fibrin glue 182 may be allowed to harden. As shown in FIG. 9A, the balloon 160 is deflated and the first forming device 158 is removed from the joint space, leaving a mold to form a neo-femoral head. The gap 184 between the acetabular component 150 and the neo-femoral head mold allows motion therebetween.

FIGS. 9B-9D show a second forming device 188 is inserted into the pilot hole 131 into the joint space 186. The head 216 of the second forming device 188 may sit on the acetabulum fossa. The first forming device 158 has two channels 202 and 204. The first channel 202 communicates with a first inlet 196 and at least two first outlets 206. The first outlets 206 are located inside the balloon 190. The second channel 204 communicates with a second inlet 198 and second outlets 208 and 210. The outlets 208 and 210 are located adjacent to the external surface of the balloon 190 and adjacent to the head 216 of the second forming device 188.

As shown in FIG. 9C, the second forming device 188 has a smaller diameter balloon 190 compared the balloon 160 of the first forming device 158. A syringe 214 is connected to the first inlet 196 to provide a fluid (e.g., air, a saline solution, etc.) for inflating the balloon 190. The balloon 190 may be inflated to a diameter such that there is a gap (or space) 192 (e.g., 1 mm) between the balloon 190 and the cured fibrin glue 182. The purpose of the smaller diameter balloon 190 is to inject a polymer 194 into the gap 192 (see FIG. 9D). The polymer 194 may include cartilage-forming cells and growth factors. The polymer 194 is configured to set on the fibrin glue and hold its shape or configuration. A syringe 218 containing polymer 194 may be connected to the second inlet 198 to provide the polymer 194 inside the second channel 204. The outlets 208 and 210 receive the polymer 194 from the second channel 204 and inject the polymer 194 inside the gap 192. The process may be conducted fluoroscopy or arthroscopy. As shown in FIG. 9E, the balloon 190 is deflated and the second forming device 188 is removed from the pilot hole 131.

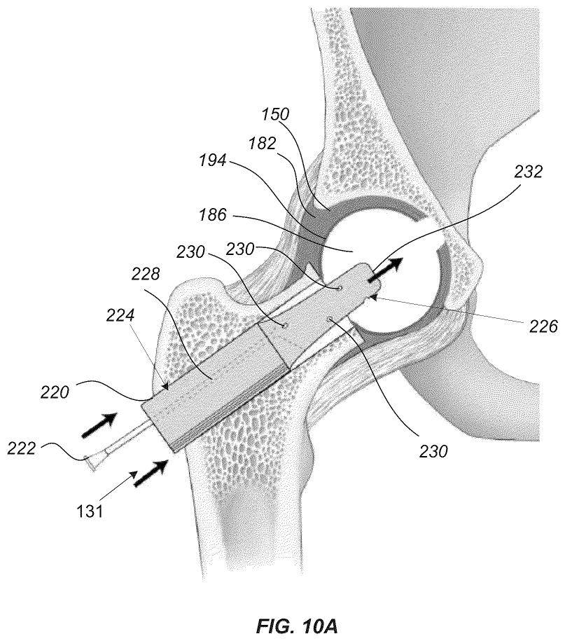

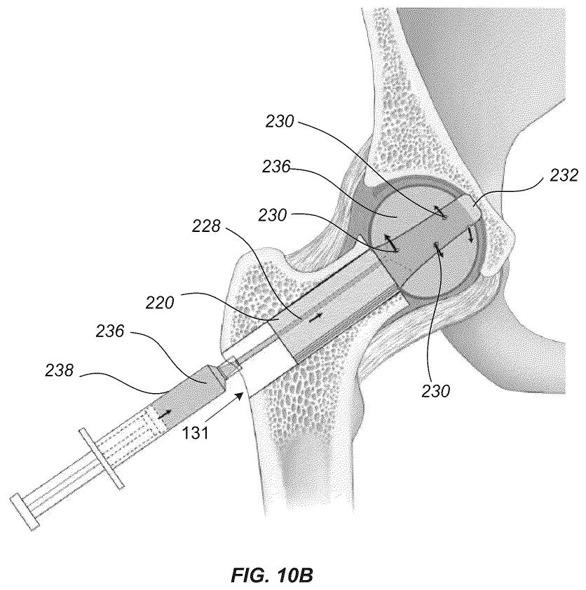

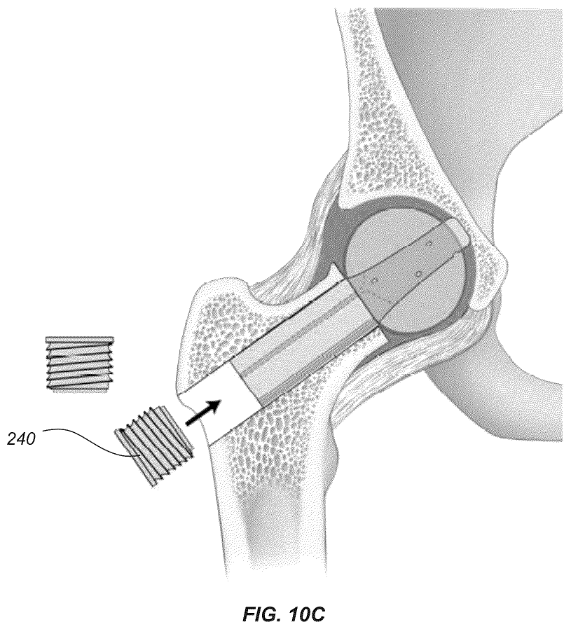

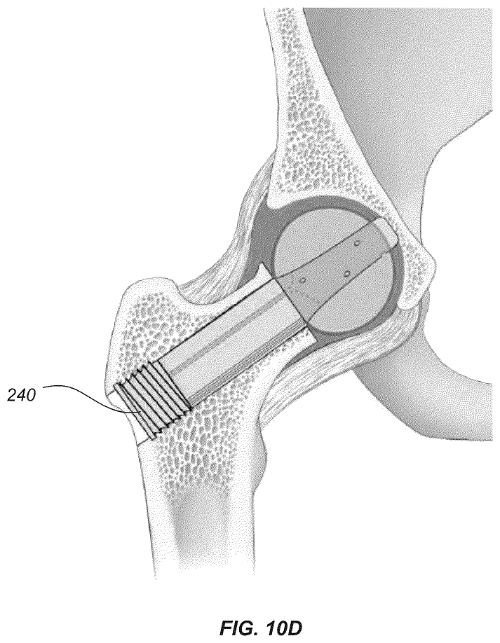

As shown in FIGS. 10A-10D, an implantable joint replacement device 220 is inserted through the pilot hole 131. The head 232 of the implantable joint replacement device 220 is docked at the acetabulum fossa. The implantable joint replacement device 220 has an elongated body. The elongated body has a lateral component 224 and a medial component 226. An internal channel 228 is formed inside the elongated body. The internal channel 228 extends along a portion of the elongated body. The implantable joint replacement device 220 has an inlet 222 adjacent to the lateral component 224 of the elongated body. The inlet 222 is in fluid communication with the internal channel 228. The implantable joint replacement device 220 has one or more outlets 230 disposed adjacent the medial end along a surface of the elongated body. The outlets 230 are in fluid communication with the internal channel 228.

In some embodiments, the elongated body can include comprises any one of: an Ossio material; an absorbable metal comprising magnesium, calcium, zinc or supplemental material; or a fully dense hydroxyapatite. The fully dense hydroxyapatite may be biologically or synthetically derived.

As shown in FIGS. 10A and 10B, a syringe 238 may be connected to the inlet 222 to inject a flowable and absorbable hydroxy-appetite scaffolding material 236 to the joint space 186. The scaffolding material 236 flows through the channel 228 and exits at the outlets 230 to settle inside the joint space 186. After injecting the scaffolding material 236, the syringe is disconnected from the inlet 222 and a compression cap 240 is inserted inside the pilot hole 131 to seal the implantable joint replacement device 220 (see FIGS. 10C and 10D). [to add more]

The scaffolding material may include calcium phosphate cement, polyethylene glycol diacrylate, cartiform allograft, polycaprolactone, thrombin and/or fibrinogen. In some embodiments, the scaffolding material 236 may include any one of: at least one cytokine; at least one growth factor; bone marrow aspirate concentrate (BMAC); dexamethasone; osteogenic protein-1 (OP-1); TGF-beta 3; recombinant human bone morphogenetic protein-7 (rhBMP-7); and/or cultured cells. For example, the scaffolding material 236 may be pre-loaded with cytokines or small molecules to facilitate local stem cell migration into the scaffold and eventual bone formation. In some embodiments, the size and spherical nature of the femoral head places some of the material at a substantial distance from the cut surface of the cancellous bone of the femoral neck. It may be desired to create a gradient of growth factors with the highest concentration furthest from the osteotomy site. This may be accomplished by mixing separate batches of the injectable material and applying them sequentially.

In some embodiments, once the cartilage layer has been formed in the shape of an egg shell, a “shape memory polymer” scaffold may be used to form the femoral head. The scaffold material may be preloaded with cytokines or cultured osteoblasts to form the bone portion of the femoral head. The scaffold material may be heated to 140 degrees F. outside of the patient. This process may provide a sponge-like and pliable scaffold material. The scaffold material may be inserted through the pilot hole into the joint space and allowed to cool to body temperature (e.g., 98.6 F). The scaffold material may be covered with polydopamine to enhance cell adherence. Once cooled, the scaffold material may resume its original configuration, which is to match the size of the bone component of the femoral head.

### Second Generation Device

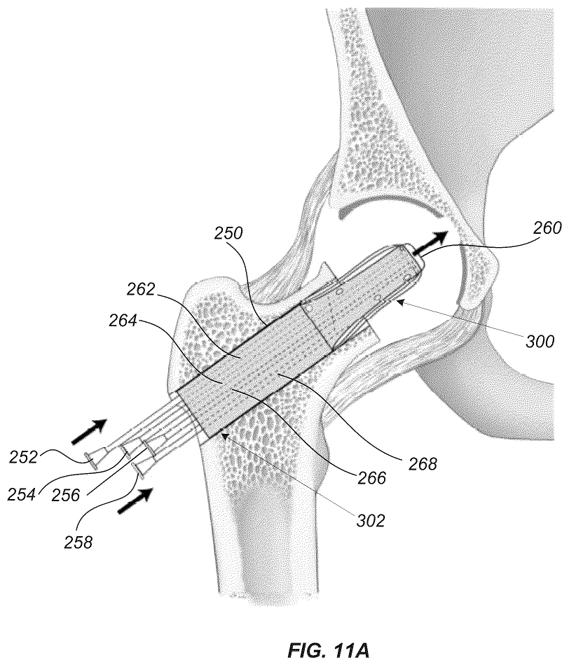

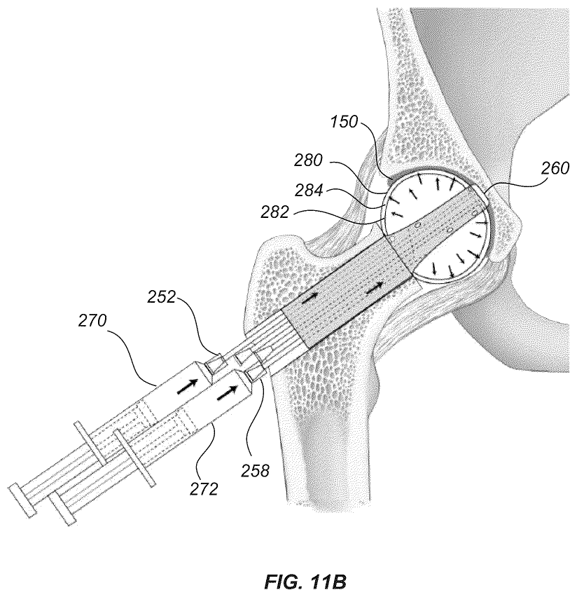

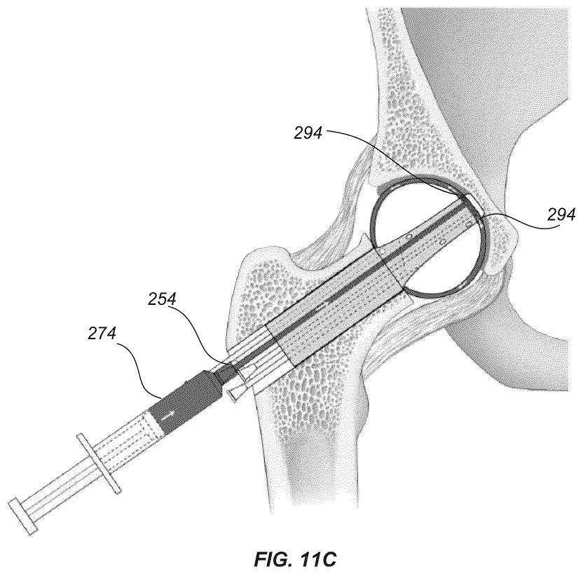

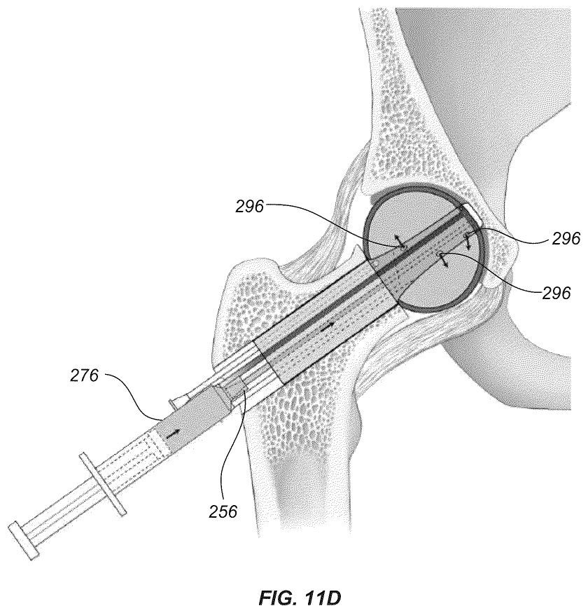

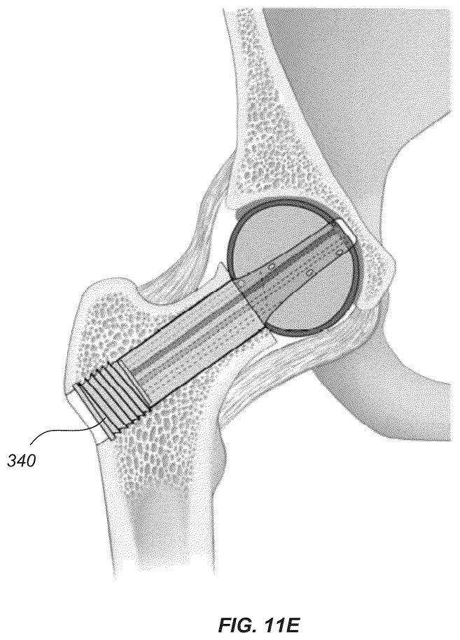

In some embodiments, once the acetabular component is deployed inside the joint space as shown in FIG. 6C, an implantable joint replacement device as shown in FIGS. 11A-11E may be mounted inside the joint space. For example, the device may include bio-absorbable balloons made from a form of polyhydroxy-alkanoate. The device may have a balloon within a balloon construct and may be inserted in the joint space in one step. Each one of the balloon may be filled, starting from the smallest balloon, and the space between the two balloons after. This may obviate the need to use fibrin glue to create a mold as previously illustrated in FIG. 8. This may be possible because fibrin glue is broken down in the joint by the plasminogen pathway.

Referring now to FIGS. 11A-11E, an implantable joint replacement device 250 includes an elongated body, an outer bioabsorbable balloon 280, an inner bioabsorbable balloon 282, a first inlet 254, a first channel 264, one or more first outlets 294, a second inlet 256, a second channel 266 and one or more second outlets 296. The head 260 of the implantable joint replacement device 250 is docked at the acetabulum fossa (see FIG. 11B). The elongated body has a medial component 300 and a lateral component 302.

The outer bioabsorbable balloon 280 and the inner bioabsorbable balloon 282 are adjacent to the medial component 300 of the elongated body. The outer balloon 280 is provided over the inner balloon 282. The first channel 264 and the second channel 266 are formed inside the elongated body and extend along a portion of the elongated body. In some embodiments, the inner and outer balloons surround a portion of the medial component 300 of the elongated body.

The first inlet 254 and the second inlet 256 are adjacent to the lateral component 302 of the elongated body. The first inlet 254 is in fluid communication with the first channel 264. The second inlet 256 is in fluid communication with the second channel 266. The first outlets 294 are in fluid communication with the first channel 264. The first outlets 194 are disposed adjacent to an external surface of the inner balloon 282 and is configured to provide a first fluid in the gap 284, which is between an internal surface of the outer balloon 280 and the external surface of the inner balloon 282 (see FIG. 11C). A syringe 274 may be connected to the first inlet 254 to provide, through the first channel 265 and first outlets 294 to provide the first fluid in the gap 284. The second outlets 296 are in fluid communication with the second channel 266. The second outlets 296 are disposed inside the inner balloon 282 and are configured to provide a second fluid inside the inner balloon (see FIG. 11D). A syringe 276 may be connected to the second inlet 256 to provide the second fluid inside the inner balloon through the second channel 266 and second outlets 296.

The implantable joint replacement device 250 may also include a third inlet 252, a third channel 262, a third outlet (not shown). The implantable joint replacement device 250 may further include a fourth inlet 258, a fourth channel 268 and a fourth outlet (not shown). The third and fourth inlets may be used to inflate the inner and outer balloons respectively by injecting fluids (e.g., air, etc.) inside them. Syringes 270 and 272 may be connected to the third and fourth inlets respectively to inject the fluids.

The elongated body may include any one of: an Ossio material; an absorbable metal comprising magnesium, calcium, zinc, or a supplemental material; or a fully dense hydroxyapatite. The fully dense hydroxyapatite may be biologically or synthetically derived.

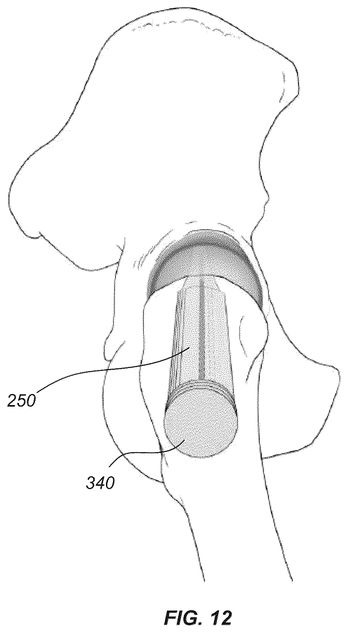

A flowable and absorbable hydroxy-appetite scaffolding material may be injected through any one of the first inlet and the second inlet. The scaffolding material may include calcium phosphate cement, polyethylene glycol diacrylate, cartiform allograft, polycaprolactone, thrombin and/or fibrinogen. In some embodiments, the scaffolding material 236 may include any one of: at least one cytokine; at least one growth factor; bone marrow aspirate concentrate (BMAC); dexamethasone; osteogenic protein-1 (OP-1); or recombinant human bone morphogenetic protein-7 (rhBMP-7). After injecting the scaffolding material, a compression cap 340 is inserted inside the pilot hole 131 to seal the implantable joint replacement device 250 (see FIG. 11E). FIG. 12 shows a view of the device 250 mounted inside the pilot hole 131.

### Multi-Step Procedure for Installing the First Generation Implant

In some embodiments, a method of performing hip replacement on a patient is provided below. The method may include separating a femoral head from a femur in a patient's body and removing the femoral head from the patient's body such that said removal forms a cavity inside the patient's body. The method may include positioning an acetabular component inside the cavity to form a cartilage layer. The acetabular component may include a material attracting stem cells to turn into cartilage. The method may include positioning and inflating a first balloon in the cavity, and injecting fibrin glue inside the cavity through a channel of the balloon. The fibrin glue may be allowed to harden for forming a mold or potential space for the neo-femoral head. The method may include deflating and removing the first balloon from the cavity. The method may include positioning and inflating a second balloon inside the space created by the fibrin glue.

The second balloon may be smaller than the first balloon such that the difference in diameters between the first and second balloons creates a desired space for materials intended to develop into cartilage. The desired space may provide motion between the acetabular component and the neo-femoral head. The method may include providing, by way of the second balloon, the cartilage materials within the space created by the difference in diameters between the first and second balloons. The method may include deflating and removing the second balloon from the cavity. The method may include positioning the first generation implantable device 220 (as shown in FIG. 10A-10D) inside the space left by the second balloon. The method may include injecting a flowable and absorbable hydroxy-appetite scaffolding material through the inlet of the implantable device 220 to form a neo-femoral head when the scaffolding material hardens into the shape of the neo-femoral head.

The method may include removing acetabular cartilage, osteophytes and/or any obstructive material after the removal of the femoral head. This removal allows for clearer access to the acetabulum to provide correct placement of a potential acetabular component. The method may include positioning the acetabular component inside the space within the acetabulum to form the cartilage layer by using an adhesive and positioning the acetabular component to the space within the acetabulum. For example, the adhesive may be fibrin glue, which is applied to an external surface of the acetabular component. The method may include aligning the acetabular component with the neo-femoral head to achieve optimal joint biomechanics and postoperative functionality to ensure that the patient has a good range of motion post-operation and reduce the chances of dislocation of the new joint.

### Single-Step Procedure for Installing the Second Generation Implant

In some embodiments, a method of performing hip replacement on a patient is provided. The method may include separating a femoral head from a femur in a patient's body and removing the femoral head from the patient's body such that said removal forms a cavity inside the patient's body. The method may include removing an acetabular cartilage, osteophytes and/or any obstructive material after the removal of the femoral head creating a space within the acetabulum and positioning an acetabular component inside the space within the acetabulum to form a cartilage layer.

The method may include positioning the second generation implantable device 250 (see FIGS. 11A-11E) inside the cavity. The method may include inflating inner and outer balloons of the implantable device 250. The method may include injecting cartilage materials through the first inlet of the implantable device into the space between the inner and outer balloons. The method may include injecting a flowable and absorbable hydroxy-appetite scaffolding material through a second inlet of the implantable device inside the inner balloon to form the neo-femoral head when the scaffolding material hardens.

### Fracture and Removal of an Osteotomized Femoral Head of a Hip Joint

In some embodiments, a method of fracturing and removing an osteotomized femoral head of a hip joint during a hip arthroplasty procedure is provided. The method may include providing stress risers on the femoral head. The stress risers may be designed to weaken the femoral head at pre-determined locations. In a first approach, the stress risers may be provided in the femoral head by guiding the arthroscopic bur to the femoral head with a fluoroscope and creating the stress risers in the femoral head using the bur. In a second approach, the stress risers may be provided in the femoral head by guiding the arthroscopic drill to the femoral head with the fluoroscope and creating the stress risers in the femoral head using the drill. In a third approach, the stress risers may be provided in the femoral head by providing a laser surgical system and a fluoroscope, guiding the laser surgical system to the femoral head with the fluoroscope and creating the stress risers in the femoral head with the laser surgical system. In a fourth approach, the stress risers may be provided in the femoral head by providing a fluoroscope and a sharp awl or pick, the awl or pick designed for use in microfracture techniques, guiding the awl or pick to the femoral head with the fluoroscope and creating multiple stress risers in the femoral head with the awl or pick by generating small fractures or punctures in the femoral head. In a fifth approach, the stress risers may be provided in the femoral head by providing a bone chisel or osteotome and a fluoroscope, guiding the bone chisel or osteotome to the femoral head with the fluoroscope and creating the stress risers in the femoral head with the bone chisel or osteotome.

The method may include positioning an inflatable bone tamp into the femoral head. In a first approach, the inflatable bone tamp may be inserted into the femoral head by providing a cannulated inflatable bone tamp, a guide wire, and a fluoroscope, inserting the guide wire into the femoral head using fluoroscopic guidance, threading the cannulated inflatable bone tamp over the guide wire so that it enters the femoral head, adjusting the inflatable bone tamp within the femoral head and optionally, removing the guide wire from the femoral head. In a second approach, the inflatable bone tamp may be inserted into the femoral head by providing a fluoroscope, under fluoroscopic guidance, navigating a site of the femoral head, inserting the inflatable bone tamp directly into a pre-prepared hole in the femoral head, the insertion conducted using real-time guidance from the fluoroscope for precision and positioning the inflatable bone tamp within the femoral head.

In a third approach, the inflatable bone tamp may be inserted into the femoral head by providing a trocar and a fluoroscope, introducing the trocar into the femoral head by way of fluoroscopic guidance, threading the inflatable bone tamp over the trocar, so that it is guided into the femoral head; adjusting the inflatable bone tamp within the femoral head and removing the trocar such that the inflatable bone tamp remains correctly positioned within the femoral head. In a fourth approach, the inflatable bone tamp may be inserted into the femoral head by providing a surgical drill, establishing access to the femur via a limited incision, generating a surgical tunnel within the femur with the surgical drill, advancing the bone tamp through the surgical tunnel and into the femoral head with guidance from fluoroscopic imaging and positioning the inflatable bone tamp within the femoral head.

The method may include inflating the bone tamp to cause a controlled fracture of the femoral head at the locations of the stress risers. In one approach, the bone tamp is inflated to cause the controlled fracture of the femoral head at the locations of the stress risers by: inflating said bone tamp using a syringe, such that the syringe is filled with a fluid and the quantity of fluid and pace of injection are controlled to regulate the speed and degree of inflation; applying outward pressure via the inflation of said bone tamp on the predetermined stress riser locations within the femoral head to induce fractures; monitoring the fracturing process using real-time imaging wherein the inflation can be paused or modified based upon the progression of the fractures; and deflating and removing said bone tamp once the femoral head is fractured into desirable pieces via the controlled fracturing, wherein said method allowing for precise fracturing of the femoral head.

The method may include removing the fractured pieces of the femoral head from the hip joint. In one approach, the fractured pieces of the femoral head are removed from the hip joint by providing a surgical forceps or a similar instrument, identifying and isolating individual fractured pieces of the femoral head within the hip joint via near real-time imaging and extracting each isolated fractured piece of the femoral head using the surgical forceps. In a further approach, the fractured pieces of the femoral head are removed from the hip joint by: providing an orthopedic burr or a similar rotary cutting tool; providing near real-time imaging to pinpoint the location of the fractured pieces within the hip joint; deploying the orthopedic burr to carefully grind or scrape away said fractured pieces; removing fractured pieces with the burr until the hip joint is cleared of all fractured pieces; and using an irrigation and suction process to remove residual debris and ensure complete clearance of the joint.

### Preoperative Planning

Preoperative planning for all joint arthroplasties may be desired. In some embodiments, this step may include determining the size of the components that will be assembled intra-operatively. This part of the preoperative planning may take part similarly to the current process of producing patient specific cutting guides (e.g., Biomet Signature Series). MRI's/CT's are performed of the patients hip and 3-D reconstructions are produced to determine the size of the femoral head, the acetabulum, the neck length and diameter of the femoral neck at the planned level of the osteotomy. This process may determine the length and the maximal diameter of the structural component. The patient's body habitus may be determined as well as their weight measured. Patients over a certain weight or with compromised access to their hip joint secondary to previous incisions, deformity, large musculature, anatomic variants or large fat distributions may be referred for standard arthroplasty. The patient's weight may determine the minimal diameter of the structural component. This minimum may be based on load to failure testing and metrics based on this data derived from in vitro proof studies of the construct. It is anticipated that once the proposed femoral structural components are determined (as opposed to the acetabular side) the surgeon may be supplied for potential implantation structural components of similar lengths and different diameters to take into account intra-operative variation from pre-op studies.

It is also recognized that there is variability that exists between individuals and within age categories that may influence the patient's stem cell response to the applied growth factors or small molecules. A pre-op test may be developed to gauge this prior to the procedure and place patients in certain subgroups stratified for healing potential. This in turn may determine the concentrations or gradients that may be used in each case to ensure component incorporation.

In some embodiments, the procedure is described as a minimally invasive surgical procedure that, in one iteration, can be done with simultaneous and arthroscopic assistance. The use of intra-operative surgical navigation in concert arthroscopy and fluoroscopy may add an additional level of accuracy to the procedure. Similarly, the use of a fluoroscopic templating device (e.g., OrthoGrid) may be used to provide additional objective, confirmatory visual cues for the surgeon and enhance the ease and reproducibility of the procedure.

In some embodiments, the scaffolding material may be delivered using through a structural component made of a nano-structured hydroxy-appetite component (e.g., nanoss structural) that is bio-absorbable albeit at a potentially different rate than that of the injectable scaffolding material.

In some embodiments, the non-structural scaffolding may be made of a polymerizable, biodegradable cement, which may be delivered into the device by means of internal conduits that are formed in the device itself. The material may be flowable such that it can be injected through a small portal. It may have the characteristic of setting in a short period of time, either being a euthermic, minimally exothermic or endothermic reaction. The material may be mixed at the time of injection as a labile powder and then the appropriate growth factor may be added. There may be wide variations in the concentration of the cytokines (if cytokines are used), which are mixed with the labile powder. Other growth factors may be used alone or in combination. In the instance of TGFβ, this may be anywhere from 10 micrograms/gram of scaffold to 3,000 micrograms/gram. Because different materials elute growth factors or small molecules at different rates and under different conditions, studies may be conducted to determine the optimal dose for a specific patient or class of patients based on available parameters. It is also possible to add growth factors or cytokines through the use of microencapsulated spheres. SDF-1 (stromal cell-derived factor 1) or SDGF-1 may be added to the mixture in such a manner. Depending on the method by which the capsules are made, the factors may elute at different rates. Concentrations of SDF-1 may vary widely anywhere from 0.002 up to 2,000 micrograms per gram of scaffold.

The material may form inter-connected channels exhibiting a porosity of sufficient size to allow the ingress of local mesenchymal stem cells which are attracted to populate the scaffolding by nature of the presence of chemotactic factors introduced at the time the scaffolding material is prepared. Once injected, the cement may then fill a bioabsorbable balloon in one iteration. If a bioabsorbable balloon (hydroxy-alkanoate) is used, the balloon may remain in place and serve as a forming mold for the femoral head and an inverse mold for the acetabular component. It will also serve as a boundary layer between the newly formed femoral head articular cartilage and the newly formed acetabular cartilage (e.g., see FIGS. 11A-11E). Once these components are formed, it may be anticipated that the material from which the balloon is formed may resorb in a biologically neutral process, e.g., hydrolysis without acid formation.

It may also be anticipated that the acetabular component could be formed with a separate step. This may include either of inserting a pre-formed mold designed to fill the acetabular space with a cut out or defect where the cartilage is not formed. The injectable material (e.g., fibrin glue) may then be delivered and once dried, the mold may be removed. Alternatively, a pre-formed structure in the shape of the acetabular cartilage (e.g., cartiform or poly-caprolactone epsilon) may be pre-loaded with cytokines and delivered through the pilot hole and cemented in place with fibrin glue (e.g., see FIGS. 6A-6C). This may be placed under arthroscopic guidance or by pre-registering the acetabular points using image guided navigation systems and delivering the material to pre-registered location.

The next step may involve the formation of the femoral head. In one iteration a balloon may be initially inflated in the joint space. The balloon may have a cylindrical rod that runs through its diameter and exits the balloon, docking in the cotyloid notch. The joint space may be filled with fibrin glue by placing a needle percutaneously under either fluoroscopic or ultrasonic guidance or by injecting the fibrin glue through a preformed access channel embedded in the guiding rod that exits in the joint space. The fibrin glue may then be injected into the joint space and then may be allowed to harden (e.g., see FIGS. 7 and 8). The balloon may then be deflated and the balloon-rod template may be removed. This may then leave a “mold” or potential space into which the neo-femoral head may be formed. This parallels the historic “lost wax method.” A second smaller balloon that is again bisected by a cylindrical rod may then be introduced into the potential space with the rod docking into the pilot space left by the first inflation. The difference in diameters between the first and second balloons may account for the space to be occupied by material that may be introduced into the space with an intended cartilage lineage. Once the second balloon has been inflated and the rod docked, the templates may be checked under fluoroscopy. Their correct positions having been confirmed, the matrix material that has been premixed with the appropriate growth factors to ensure a cartilage lineage develops may be introduced through preformed portals in the rod-balloon construct. The second balloon and rod construct may then be deflated and removed. This may then leave the final potential space mold for the bone phase of the neo-femoral head (e.g., see FIGS. 9A-9D). The structural rod (e.g., Nanoss) is then introduced and docked into the pilot hole created by the first two steps. Once this is confirmed, the remaining space may be filled with the injectable polymerizable cement that has been pre-loaded with a dexamethasone mixture to ensure that it becomes bone (see for e.g., FIGS. 10A-10D). An alternative version may employ a balloon molding device that is made of a polyhydroxyalkanoate material. This may require the second balloon to be inflated first.

The insertional step including placement of the structural rod with the balloon component attached involves delivering the rod construct through the pilot hole. It may be anticipated that the rod would be 0.5-1.0 mm greater in diameter than the pilot hole thereby creating an interference fit between the rod and the inner cortex of the femoral neck. However, it may be desirable to retain as much of the femoral metaphyseal-neck junctional cancellous bone as possible as this is a rich repository of stem cells. Thus, the thinnest diameter structural component that may be used in any one patient would be optimal for regenerative purposes while still retain sufficient structural strength. This strength of the component would be ascertained from mechanical studies and would correlate with the patients weight, neck length, shaft angle and offset. This may be pre-operative determined by an algorithm. It may be likely that redundancy would be built into this metric to allow two standard deviations greater strength than is minimally required. As such, it may be the case that the pilot hole may be less than the minimum diameter of the neck and therefore the structural component would not have an interference fit with the cancellous bone of the femoral neck but rather would have a surrounding cuff of cancellous bone. The end of the which is the lead end, i.e., that which is inserted first may not be flat such as a cut end of a cylinder, rather it would have curvature which would match that of the newly formed femoral head. The device may be based on a central cylinder, which may be fabricated from: a nano-structured hydroxy-appetite (e.g., Nanoss); an Ossio material; or an absorbable metal comprising magnesium, calcium, zinc, or a supplemental material.

This may serve the function of supporting the construct while the scaffolding matures into functional tissue. Once this has occurred (the amount of time for the scaffolding to be sufficiently matured to self-sustaining in full weight bearing activities will be based on histology and mechanical testing determined by R&D), the structural component may subside back into the pilot track and be either level with the articular cartilage or slightly recessed. The structural rod may act as a piece of “biologic rebar” to off load the shear forces which the newly formed femoral head may experience while it is developing mature bone. A mechanism for the controlled subsidence pattern of the structural rod may allow for the initial load bearing then load-sharing role of the structural rod. This may be accomplished by the insertion of a resorbable screw inserted at the entrance portal just distal and recessed behind the rod. As the screw is absorbed and weakens, it may allow the rod to slowly back down the pilot hole and thus gradually transfer load to the new construct. It may be possible that in growth may occur between the injectable biomaterial and the structural rod (or elongated body). This could result in the inability of the rod to “back out” at the optimal time to allow increased load sharing.

It is possible that during the initial osteotomy of the femoral neck, depending on the level of the resection and the extent of femoral head resection, the blood supply to the femoral head may be interrupted or compromised. This may potentially occur by injury to the epiphyseal arteries. However, it may be shown in other applications that the growth factors used in the procedure are angiogenic so we anticipate that a new blood supply would be established by new vascularization formed through the porous scaffolding. Neo-vascularization may occur through the injection channels in the structural component. This may be facilitated by filling the channel(s) that exit into the femoral head balloon with micro spheres containing an angiogenic-specific growth factor (e.g., VEGF). For example, this may be done at the last step such that unlike FIG. 10D, the channels may be cleared at the end of the injection process by injecting saline and then the channels may be filled with angiogenic factor of choice.

During pre-op planning, the patients weight may be obtained, preferable within two weeks of the procedure. The joint reaction force may then be calculated using free body diagram. The standard formula would be used:

$${R}_{y} = {M}_{y} + W$$

where Ry=joint reaction force,
   M=abductor muscle force,
   W=gravitational force,
      weight of body minus weight of ipsilateral extremity (or ⅚ body weight).

This may be the amount of distraction force that may be applied across the structural component at the time of implantation. An additional force of 10% may be added to account for the documented average weight gain of patients following elective hip surgery.

It may be possible that a load cell with telemetry capacity may be incorporated as part of the construct, placed at the distal end of the structural component. The screw may then compress against the load cell. This may provide real time remote monitoring of the patient's weight bearing load as seen by construct. This may be recorded and used to help modify the patient's activity and even linked with an app that may alert the patient if they were exceeding the construct's desired load profile. The data may also be used for future device planning and development. As part of the preoperative counseling of the patient, permission may be obtained to gather, store, monitor and analyze this data.

Once completed the portals (or pilots holes) may be closed and images may be taken. The patient may be discharged on crutches and non-weight bearing until instructed otherwise. The patient may likely be placed on low dose ASA for a short time post-operatively to diminish the likelihood of DVT formation. The patient may be seen at pre-determined intervals post-op and examined to access their clinical progress and the progress of the device's incorporation. Once there may be evidence of radiographic bone maturation, a program of gradual, metered weight bearing may be instituted. Full weight bearing and return to full, unrestricted activity may be based on clinical and radiographic parameters.

1. An implantable joint replacement device, comprising:
an elongated body having a medial component and a lateral component;
an internal channel formed inside the elongated body, the internal channel extending along a portion of the elongated body;
an inlet adjacent to the lateral component of the elongated body, the inlet being in fluid communication with the internal channel; and
an outlet disposed along a surface of the elongated body, the outlet being in fluid communication with the internal channel.

2. The implantable joint replacement device of claim 1, wherein the elongated body comprises any one of:
an Ossio material;
an absorbable metal comprising magnesium, calcium, zinc, or a supplemental material; or
a fully dense hydroxyapatite.

3. The implantable joint replacement device of claim 2, wherein the fully dense hydroxyapatite is biologically or synthetically derived.

4. The implantable joint replacement device of claim 1, further comprising a flowable and absorbable hydroxy-appetite scaffolding material, wherein the scaffolding material is injectable through the inlet.

5. The implantable joint replacement device of claim 4, wherein the scaffolding material comprises calcium phosphate cement, polyethylene glycol diacrylate, cartiform allograft, polycaprolactone, thrombin and/or fibrinogen.

6. The implantable joint replacement device of claim 4, wherein the scaffolding material further comprises at least one cytokine.

7. The implantable joint replacement device of claim 4, wherein the scaffolding material further comprises at least one growth factor.

8. The implantable joint replacement device of claim 4, wherein the scaffolding material further comprises bone marrow aspirate concentrate (BMAC).

9. The implantable joint replacement device of claim 4, wherein the scaffolding material further comprises dexamethasone.

10. The implantable joint replacement device of claim 4, wherein the scaffolding material further comprises osteogenic protein-1 (OP-1).

11. The implantable joint replacement device of claim 4, wherein the scaffolding material further comprises recombinant human bone morphogenetic protein-7 (rhBMP-7).

12. An implantable joint replacement device, comprising:
an elongated body having a medial component and a lateral component;
an outer bioabsorbable balloon and an inner bioabsorbable balloon, both inner and outer balloons adjacent to the medial component of the elongated body, the outer balloon being provided over the inner balloon;
a first channel and a second channel, the first and second channels formed inside the elongated body and extending along a portion of the elongated body;
a first inlet and a second inlet, both adjacent to the lateral component of the elongated body, the first inlet being in fluid communication with the first channel and the second inlet being in fluid communication with the second channel;
a first outlet in fluid communication with the first channel, the first outlet being disposed adjacent to an external surface of the inner balloon and configured to provide a first fluid between an internal surface of the outer balloon and the external surface of the inner balloon; and
a second outlet in fluid communication with the second channel, the second outlet being disposed inside the inner balloon and configured to provide a second fluid inside the inner balloon.

13. The implantable joint replacement device of claim 12, wherein the inner and outer balloons surround a portion of the medial component of the elongated body.

14. The implantable joint replacement device of claim 12, wherein the elongated body comprises any one of:
an Ossio material;
an absorbable metal comprising magnesium, calcium, zinc, or a supplemental material; or
a fully dense hydroxyapatite.

15. The implantable joint replacement device of claim 14, wherein the fully dense hydroxyapatite is biologically or synthetically derived.

16. The implantable joint replacement device of claim 12, further comprising a flowable and absorbable hydroxy-appetite scaffolding material, wherein the scaffolding material is injectable through any one of the first inlet and the second inlet.

17. The implantable joint replacement device of claim 16, wherein the scaffolding material comprises calcium phosphate cement, polyethylene glycol diacrylate, cartiform allograft, polycaprolactone, thrombin and/or fibrinogen.

18. The implantable joint replacement device of claim 16, wherein the scaffolding material further comprises at least one cytokine.

19. The implantable joint replacement device of claim 16, wherein the scaffolding material further comprises at least one growth factor.

20. The implantable joint replacement device of claim 16, wherein the scaffolding material further comprises bone marrow aspirate concentrate (BMAC).

21. The implantable joint replacement device of claim 16, wherein the scaffolding material further comprises dexamethasone.

22. The implantable joint replacement device of claim 16, wherein the scaffolding material further comprises osteogenic protein-1 (OP-1).

23. The implantable joint replacement device of claim 18, wherein the scaffolding material further comprises recombinant human bone morphogenetic protein-7 (rhBMP-7).

24. A method of performing hip replacement on a patient, comprising:
separating a femoral head from a femur in a patient's body;
removing the femoral head from the patient's body such that said removal forms a cavity inside the patient's body;
positioning an acetabular component inside the cavity to form a cartilage layer, wherein the acetabular component comprises a material attracting stem cells to turn into cartilage;
positioning and inflating a first balloon in the cavity, and injecting fibrin glue inside the cavity through a channel of the balloon, wherein the fibrin glue is allowed to harden for forming a mold or potential space for a neo-femoral head;
deflating and removing the first balloon from the cavity;
positioning and inflating a second balloon inside the space created by the fibrin glue, the second balloon being smaller than the first balloon such that the difference in diameters between the first and second balloons creates a desired space for materials intended to develop into cartilage, wherein the desired space provides motion between the acetabular component and the neo-femoral head;
providing, by way of the second balloon, the cartilage materials within the space created by the difference in diameters between the first and second balloons;
deflating and removing the second balloon from the cavity;
positioning the implantable device of claim 1 inside the space left by the second balloon; and
injecting a flowable and absorbable hydroxy-appetite scaffolding material through the inlet of the implantable device to form a neo-femoral head when the scaffolding material hardens into the shape of the neo-femoral head.

25. The method of claim 24, further comprising:
removing acetabular cartilage, osteophytes and/or any obstructive material after the removal of the femoral head, wherein said removal allows for clearer access to the acetabulum to provide correct placement of a potential acetabular component.

26. The method of claim 25, wherein positioning the acetabular component inside the space within the acetabulum to form the cartilage layer comprises:
applying an adhesive, and, optionally fibrin glue, to an external surface of the acetabular component; and
positioning the acetabular component to the space within the acetabulum.

27. The method of claim 26, further comprising aligning the acetabular component with the neo-femoral head to achieve optimal joint biomechanics and postoperative functionality to ensure that the patient has a good range of motion post-operation and reduce the chances of dislocation of the new joint.

28. A method of performing hip replacement on a patient, comprising:
separating a femoral head from a femur in a patient's body;
removing the femoral head from the patient's body such that said removal forms a cavity inside the patient's body;
removing an acetabular cartilage, osteophytes and/or any obstructive material after the removal of the femoral head creating a space within the acetabulum;
positioning an acetabular component inside the space within the acetabulum to form a cartilage layer;
positioning the implantable device of claim 12 inside the cavity;
inflating inner and outer balloons of the implantable device;
injecting cartilage materials through the first inlet of the implantable device into the space between the inner and outer balloons; and
injecting a flowable and absorbable hydroxy-appetite scaffolding material through a second inlet of the implantable device inside the inner balloon to form the neo-femoral head when the scaffolding material hardens.

29. A method of fracturing and removing an osteotomized femoral head of a hip joint during a hip arthroplasty procedure, the method comprising:
providing stress risers on the femoral head, the stress risers designed to weaken the femoral head at pre-determined locations;
positioning an inflatable bone tamp into the femoral head;
inflating the bone tamp to cause a controlled fracture of the femoral head at the locations of the stress risers; and
removing the fractured pieces of the femoral head from the hip joint.

30. The method of claim 29, wherein providing stress risers in the femoral head comprises:
providing a fluoroscope and an arthroscopic bur;
guiding the arthroscopic bur to the femoral head with the fluoroscope; and
creating the stress risers in the femoral head using the bur.

31. The method of claim 29, wherein providing stress risers in the femoral head comprises:
providing a fluoroscope and an arthroscopic drill;
guiding the arthroscopic drill to the femoral head with the fluoroscope; and
creating the stress risers in the femoral head using the drill.

32. The method of claim 29, wherein providing stress risers in the femoral head comprises:
providing a laser surgical system and a fluoroscope;
guiding the laser surgical system to the femoral head with the fluoroscope; and
creating the stress risers in the femoral head with the laser surgical system.

33. The method of claim 29, wherein providing stress risers in the femoral head comprises:
providing a fluoroscope and a sharp awl or pick, the awl or pick designed for use in microfracture techniques;
guiding the awl or pick to the femoral head with the fluoroscope; and
creating multiple stress risers in the femoral head with the awl or pick by generating small fractures or punctures in the femoral head.

34. The method of claim 29, wherein providing stress risers in the femoral head comprises:
providing a bone chisel or osteotome and a fluoroscope;
guiding the bone chisel or osteotome to the femoral head with the fluoroscope; and
creating the stress risers in the femoral head with the bone chisel or osteotome.

35. The method of claim 29, wherein inserting the inflatable bone tamp into the femoral head comprises:
providing a cannulated inflatable bone tamp, a guide wire, and a fluoroscope;
inserting the guide wire into the femoral head using fluoroscopic guidance;
threading the cannulated inflatable bone tamp over the guide wire so that it enters the femoral head;
adjusting the inflatable bone tamp within the femoral head; and
optionally, removing the guide wire from the femoral head.

36. The method of claim 29, wherein inserting the inflatable bone tamp into the femoral head comprises:
providing a fluoroscope;
under fluoroscopic guidance, navigating a site of the femoral head;
inserting the inflatable bone tamp directly into a pre-prepared hole in the femoral head, the insertion conducted using real-time guidance from the fluoroscope for precision; and
positioning the inflatable bone tamp within the femoral head.

37. The method of claim 29, wherein inserting the inflatable bone tamp into the femoral head comprises:
providing a trocar and a fluoroscope;
introducing the trocar into the femoral head by way of fluoroscopic guidance;
threading the inflatable bone tamp over the trocar, so that it is guided into the femoral head;
adjusting the inflatable bone tamp within the femoral head; and
removing the trocar such that the inflatable bone tamp remains correctly positioned within the femoral head.

38. The method of claim 29, wherein inserting the inflatable bone tamp into the femoral head comprises:
providing a surgical drill;
establishing access to the femur via a limited incision;
generating a surgical tunnel within the femur with the surgical drill;
advancing the bone tamp through the surgical tunnel and into the femoral head with guidance from fluoroscopic imaging; and
positioning the inflatable bone tamp within the femoral head.

39. The method of claim 29, wherein inflating the bone tamp to cause the controlled fracture of the femoral head at the locations of the stress risers comprises:
inflating said bone tamp using a syringe, said syringe filled with a fluid, wherein the quantity of fluid and pace of injection are controlled to regulate the speed and degree of inflation;
applying outward pressure via the inflation of said bone tamp on the predetermined stress riser locations within the femoral head to induce fractures;
monitoring the fracturing process using real-time imaging wherein the inflation can be paused or modified based upon the progression of the fractures; and
deflating and removing said bone tamp once the femoral head is fractured into desirable pieces via the controlled fracturing, wherein said method allowing for precise fracturing of the femoral head.

40. The method of claim 29, wherein removing the fractured pieces of the femoral head from the hip joint comprises:
providing a surgical forceps or a similar instrument;
identifying and isolating individual fractured pieces of the femoral head within the hip joint via near real-time imaging; and
extracting each isolated fractured piece of the femoral head using the surgical forceps.

41. The method of claim 29, wherein removing the fractured pieces of the femoral head from the hip joint comprises:
providing an orthopedic burr or a similar rotary cutting tool;
providing near real-time imaging to pinpoint the location of the fractured pieces within the hip joint;
deploying the orthopedic burr to carefully grind or scrape away said fractured pieces;
removing fractured pieces with the burr until the hip joint is cleared of all fractured pieces; and
using an irrigation and suction process to remove residual debris and ensure complete clearance of the joint.
